(19) United States
(12) Patent Application Publication
(10) Pub. No.: US 2018/0333429 A1
(43) Pub. Date: Nov. 22, 2018

(54) FASTING FORMULATION

(71) Applicant: Medical Symbiosis Pty Ltd, Forrest (AU)

(72) Inventor: David Reiner, Forrest (AU)

(21) Appl. No.: 15/596,372

(22) Filed: May 16, 2017

(51) Int. Cl. A61K 31/718; A61K 9/00; A23L 2/60

(52) U.S. Cl. CPC A61K 31/718; A61K 9/0095; A23V 2002/00; A23L 2/60; A61K 9/0053

(57) ABSTRACT

The present invention relates to a fasting formulation and a method of use that assists a patient during a fasting period and to reduce side effects of the fasting. Furthermore, the invention promotes better recovery from fasting. The formulation contains maltodextrin and is devoid of simple sugars. The invention further relates to the use of the formulation as a pre-operative for preparing for surgery and anaesthesia using the formulation.

### BACKGROUND

### Technical Field

The present invention relates to a fasting formulation and a method of use that assists a patient during a fasting period and to reduce side effects of the fasting. Furthermore, the invention promotes better recovery from fasting. The formulation contains maltodextrin and is devoid of simple sugars. The invention further relates to the use of the formulation as a pre-operative for preparing for surgery and anaesthesia using the formulation.

### Background Information

There are many reasons why a person would choose to fast but more than often, the reason for fasting is to prepare for surgery and anaesthesia. A period of fasting may begin just before midnight for morning surgery and last to the next day even extending to the afternoon if surgery times are delayed. A fasting period may last for as long as 16 to 18 hours.

In this period, the patient will experience considerable changes in metabolism which affect the carbohydrate, protein and fat reserves of the body. The requirements of the brain for glucose must be satisfied from glycogen stored in the liver. Other additional energy sources will come from breakdown of protein in the muscle to amino acids, and fat degradation. The breakdown of energy stores can cause physical stress and trauma to the body and does not place the patient in the best condition prior to surgery. Such fasting regimes may put the body in a dehydrated and catabolic state where the body is breaking down protein and fats to provide energy. Headaches, nausea, lethargy, thirst and hunger are common as a result.

Coordinating a patient for surgery is a complex task and the routine practicalities in managing this task need to be contemplated. Hospital staff are often tasked with providing patients with fasting instructions, usually the day before surgery. During the consultation the patients are often presented with a confusing number of options in the event that they become hungry or thirsty during the fasting period. Their options can include black coffee, no milk in tea, juice but no pulp, food 6 hours before, clear fluids two hours before etc. Patients are therefore concerned about ingesting the right fluids in the event that they may die if they eat or drink anything they should not or that their surgery may be cancelled if their allocated anaesthetist finds out that they didn't comply. Patients therefore often choose to starve or not drink at all. As a result of the avoidance of suitable fluids, the patients become angry, are dissatisfied with their treatment in the hospital, disrupt the ward tranquillity and complain and become aggressive toward staff when they are hungry and thirsty.

Some patients find the options confusing and theatre times are not always guaranteed. Patients should only begin fasting from fluids when the patient has been allocated a specific theatre time. Too many elderly patients are presenting to theatre in a poor metabolic state. Their poor status is often exacerbated because they find it difficult to comply with procedures prior to surgery. Providing the patient with simple instructions prior to the operation will result in a better outcome.

During the fasting period the body may also experience the reduced effectiveness of insulin. A state of insulin resistance is developed. The degree of insulin resistance developing after surgery due to this long period of fasting is of major concern following surgery. Insulin resistance has been associated with increased length of hospital stay.

The stress of surgery additionally results in the release of stress hormones into the bloodstream (cortisol from the adrenal glands amongst other hormones). These hormones result in catabolic derangements of metabolic and physiological processes.

The catabolism that results causes muscle wasting, impaired immune function and wound healing even organ failure and death can result.

It can take approximately three weeks after routine surgery to restore the normal balance in metabolism after surgery.

However, administering solutions containing simple sugars such as fructose and glucose may contribute to insulin resistance. The general practice is to provide the patient with carbohydrate solutions including glucose and fructose to assist in the recovery from fasting.

A direct relationship exists between insulin resistance and infectious morbidity including surgical site infections (SSI). However, the addition of sugars such as fructose and glucose may exacerbate the problem. Furthermore the addition of fructose is known to cause the unpleasant abdominal side effects of bloating, cramping and diarrhoea.

Therefore a clear option to the patients in the fasting period would enable them to overcome these feelings of hunger and thirst and put the body in a better condition to commence healing and recovery and mitigate the stress response associated with surgery.

It is therefore desirable to provide a simple formulation to be introduced in a fasting period such as in a pre-operative stage to prevent starvation, allow metabolic optimisation of patients and facilitate providing a simple guideline prior to surgery which will place the patient at ease at least in regard to the stages prior to surgery and anaesthesia and to improve recovery after surgery.

### SUMMARY

Fasting prior to surgery will often induce hunger, thirst, headaches, nausea and lethargy and patients having to fast will be irritable as a consequence of extended fasting.

Accordingly, in one aspect there is provided a formulation when used to reduce side effects of fasting comprising maltodextrin and is devoid of simple sugars. The formulation of the present invention is a simple solution that contains maltodextrin in the absence of other simple sugars such as but not limited to glucose, fructose and galactose.

In another aspect there is provided a use of maltodextrin in the absence of simple sugars in the preparation of a formulation which is devoid of simple sugars to reduce side effects of fasting.

In yet another aspect of the invention there is provided a method for preparing for surgery, said method comprising:
ceasing eating for a time prior to surgery to induce fasting;
ingesting a formulation comprising maltodextrin and devoid of simple sugars during the fasting at about at least 90 minutes prior to surgery.

The invention also provides instructions for ingesting the formulation to alleviate the side effects of fasting and prepare the body for pre- and post-surgery. Patients who prepare the body with the formulation are less likely to experience post-surgery complications and recover more easily than those that do not.

Other aspects of the present invention will become apparent to those ordinarily skilled in the art upon review of the following description of specific embodiments of the invention.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

### BRIEF DESCRIPTION OF THE DRAWINGS

FIGS. 1A-E show fasting cards that can be provided to patients for the use of the formulation prior to surgery. (A) Bowel prep fasting card; (B) Morning surgery fasting card; (C) Afternoon surgery fasting card; (D) Morning surgery fasting card for one bottle and (E) Afternoon surgery fasting card for one bottle.

### DETAILED DESCRIPTION

Patients that are required to fast prior to surgery will often experience hunger, thirst, headaches, nausea and lethargy and will be irritable as a consequence of extended fasting. Such fasting regimes may put the body in a dehydrated and catabolic state, where the body is breaking down protein to provide energy. However, the options available to the patient during this period to satisfy these feelings of hunger and thirst and subdue other side effects are not always clear. Hence, the patient will often do nothing or simply drink water which does not satisfy the feelings of hunger or provide sufficient energy sources.

Accordingly in an aspect of the present invention there is provided a formulation when used to reduce side effects of fasting comprising maltodextrin and is devoid of simple sugars.

The formulation of the present invention is a simple solution that contains maltodextrin in the absence of other simple sugars such as but not limited to glucose, fructose and galactose. Because there are no simple sugars present, the formulation presents less gastrointestinal discomfort and upset whilst providing fluids and energy in the form of maltodextrin. This carbohydrate drink as a liquid meal introduced at the preoperative stage can prevent starvation and allows metabolic optimisation of patients.

The present invention is a form of prophylactic treatment, which can minimise insulin resistance that results after surgery. The complex carbohydrate drink is devoid of fructose and other simple sugars but comprises maltodextrin. Maltodextrin has a high glycaemic index which is higher than fructose causing an insulin response in the human body higher per gram of maltodextrin compared with fructose. Therefore without being limited by theory, it is postulated that the formulation of the present invention which contains maltodextrin with no simple sugars such as fructose will generate a more exaggerated insulin response from the pancreas. The higher insulin response puts the body into an anabolic mode which is desired post-surgery to help heal wounds. The formulation specifically excludes simple sugars in order to stimulate a higher insulin response. Insulin is the main anabolic hormone of the body and counteracts the stress hormone response that occurs with surgery.

Fasting is a willing abstinence or reduction from some or all food, drink, or both, for a period of time. In a physiological context, fasting may refer to the metabolic status of a person who has not eaten overnight, or to the metabolic state achieved after complete digestion and absorption of a meal. There are many reasons for fasting. However, for the purposes of the present invention, the fasting is induced preferably before surgery and anaesthesia and the formulation of the present invention is preferably intended for use as a pre-operative drink. Many people may also fast as part of a medical procedure or check-up such as a colonoscopy.

The absence of simple sugars such as glucose, fructose, maltose and galactose prevents osmolality rising and for fructose can cause symptoms of abdominal cramps, bloating and diarrhoea. This is often encountered in sports drinks and juices which are often too sweet. Gastric emptying is faster with lower osmolality drinks and rapid gastric emptying is important for achieving an empty stomach prior to anaesthesia. Simple sugars are carbohydrates that are quickly absorbed by the body to produce energy. They are also classified as “simple” because they contain only one or two units of sugar or saccharides.

The formulation of the present invention is absent or devoid of simple sugars such as glucose and fructose, more preferably fructose.

Fructose has been implicated in a number of disorders including weight gain, insulin resistance, reduced insulin sensitivity and many other carbohydrate metabolism disorders including fructose metabolism disorders, galactosemia, glycogen storage diseases and pyruvate metabolism disorders. Additionally, patients ingesting fructose can experience gastrointestinal discomfort.

Preferably, the formulation is free of fat, glucose, lactose, protein and fibre.

The use of the term “absent” or “devoid” with respect to the simple sugars in the formulation means that the simple sugars are not present in an amount which would provide any measurable or effective levels of calories which would impart an effect on the body as a result of the contributions made by the presence of the simple sugars such as glucose and fructose.

The formulation is preferably a clear fluid designed to empty rapidly from the stomach. However, the formulation may be a concentrate that can be dilute to form a beverage.

The formulation is preferably in the form of a liquid and may be further formulated into a beverage. It may be provided as a syrup which can be diluted to taste. Generally, the dilution will be with water. Alternatively, the formulation may be provided as a gel that can be dissolved in the mouth, preferably with water. However, it is contemplated that a concentrated form of the formulation may be provided which can be diluted.

Preferably the formulation is more alkaline than sports drinks and it is preferred that the formulation have a pH of greater than 4.0. Preferably, the pH is in the range of 4.0 to 5.0. More preferably the pH is 4.0, 4.1, 4.2, 4.3, 4.4, 4.5, 4.6, 4.7, 4.8, 4.9 or 5.0. Most preferably, the pH is 4.2. Increasing pH results in less damage in the event of aspiration during surgery. Acidity regulators such as sodium citrate and potassium citrate may be used to regulate the pH and minimise gastric acidity.

Any food grade acids known and available to the skilled addressee may be used to adjust the pH of the formulation. Acids such as citric acid, sodium citrate or potassium citrate, or acetic acid may be used.

The only carbohydrate in the formulation is maltodextrin. There may be other additives such as flavourings and colourings, however any carbohydrates associated with these is minimal to the point of having no effect on the body. Simple sugars such as glucose, fructose or galactose, preferably fructose are absent from the formulation.

Maltodextrin is a complex carbohydrate and may be derived from any starch such as corn or wheat. Maltodextrin is generally available and commonly used as a food additive. However, in these uses, it is often accompanied with other sugars such as glucose and fructose since maltodextrin is moderately sweet or almost flavourless on its own. Hence when used in a pre or postoperative formulation manufacturers will accompany the maltodextrin with other sweeteners.

Previously, maltodextrin has not been used on its own in a pre or postoperative formulation. As stated above, it has little or no flavour and prior formulations containing maltodextrin will have other sweeteners such as fructose and glucose to improve palatability. The formulation is designed to alleviate hunger associated with prolonged fasting and hence the formulation is preferably designed to deliver at least about 100 calories per serving. These calories may be ingested as one serving or multiple servings prior to surgery. Preferably, all of the calories are derived from maltodextrin in the formulation.

The calories may be derived from at least 50 grams of maltodextrin.

Maltodextrin may be in the form of Maltodextrin with a Dextrose Equivalent value of 15 to 19.

In a preferred formulation, there may be at least 10 percent maltodextrin up to 100 percent maltodextrin in the formulation. Preferably there is provided a formulation with 10 percent, 20 percent, 30 percent, 40 percent, 50 percent, 60 percent, 70 percent, 80 percent, 90 percent or 100 percent maltodextrin. More preferably, the maltodextrin component is 10 percent, 11 percent, 12 percent, 13 percent, 14 percent, 15 percent, 16 percent, 17 percent, 18 percent, 19 percent or 20 percent, most preferably 14 percent maltodextrin. The remaining part of the formula will comprise water, acids, acidity regulators, natural flavours and colours.

The osmolality of the formulation is best kept lower than physiological range. Preferably, the formulation is about 130 to 180 mOsm/kg, preferably the osmolality is in the range of 140 to 180 mOsm/kg. In one embodiment the osmolality is 170, 171, 172, 173, 174 or 175 mOsm/kg. More preferably, the osmolality is 174 mOsm/kg. In another embodiment, the osmolality is 148, 149, 150, 151, or 152 mOsm/kg. More preferably, the osmolality is 149 mOsm/kg. More preferably, the osmolality of the formulation is provided by the maltodextrin alone and not from other sugars such as simple sugars which will be absent from the formulation of the present invention.

Other flavourings may be used providing they do not substantially alter or contribute to the calorie content or the osmolality provided by the maltodextrin alone. Natural flavourings may be used such as but not limited to natural mango, coconut, lime, ginger, peppermint, coffee, tea, raspberry, lemon or orange.

Side effects of fasting can include hunger, thirst, headaches, nausea and lethargy as well as gastrointestinal discomfort. Patients breaking a fasting period with a carbohydrate drink often experience gastrointestinal discomfort from the ingestion of the carbohydrate drinks that are sometimes offered as pre or postoperative drinks. These contain sugars such as glucose and fructose often added to improve palatability or caloric value. However the present invention is absent these simple sugars such as glucose and fructose and preferably, the formulation of the present invention reduces the side effects of gastrointestinal discomfort.

GI discomfort may come in the form of cramps, abdominal bloating, nausea and diarrhoea.

When used during fasting the formulation can keep the body nourished, hydrated and comfortable before surgery with reduced GI discomfort. This alleviates the hunger and thirst that accompanies fasting and prevents other side effects such as headaches, light headedness, nausea and lethargy so often experienced during fasting. In the preoperative phase, the patients are in a better physical and mental state.

Continued use of the formulation after fasting and/or surgery enhances recovery and helps reduce post-surgical complications such as infection by inducing immune function and reducing insulin resistance and insensitivity. During the postoperative phase and with the use of the formula, the immune response is facilitated by minimising postoperative hyperglycaemia when excess blood sugar can inhibit phagocytosis and chemotaxis.

In another aspect of the present invention, there is provided a use of maltodextrin in the absence of simple sugars in the preparation of a formulation which is devoid of simple sugars to reduce side effects of fasting.

Maltodextrin has been used in sports drinks and pre-operative drinks. However, it has not been used alone in the absence of simple sugars to provide a formulation that reduces side effects of fasting such as hunger and thirst, preferably with reduced GI discomfort.

Applicants have found that sports drinks and pre-operative drinks can cause GI discomfort especially if consumed during and after fasting. Often they will contain simple sugars such as fructose.

Preferably the formulation, in which the maltodextrin is used, is described herein.

In yet another aspect of the present invention there is provided a method for preparing a patient for surgery and anaesthesia, said method comprising the patient:
ceasing eating for a time prior to surgery and anaesthesia to induce fasting in the patient;
ingesting a formulation comprising maltodextrin and which is devoid of simple sugars during the fasting at about at least 90 minutes prior to surgery and anaesthesia.

The formulation of the present invention is provided as a fasting formulation which can reduce side effects of fasting. It is contemplated that an ideal use is that of a preoperative formulation designed to prepare a patient for surgery and anaesthesia. Prior to surgery and anaesthesia, patients are asked to fast. Preferably this time will be from at least 12 hours prior to surgery and anaesthesia. However shorter times may be adopted providing the stomach is suitably emptied.

Patients are called or seen in preadmission clinic before surgery and anaesthesia. The patients are presented with a confusing array of multiple fasting options to assist them through the fasting period including black coffee, no milk in tea, apple juice, no pulp, food 6 hours before, clear fluids two hours before etc., whereby the patient comprehends that basically they might die if they eat or drink anything, and that their surgery will be cancelled if their allocated surgeon or anaesthetist finds out they didn't comply.

Complete oral abstinence is presented as being equivalent, if not more superior to having a liquid meal. As a result of this, patients starve themselves in an attempt to be “more safe”. This practice harms the patient and leaves them in a less prepared state for surgery and jeopardises their recovery following surgery.

Preferably prior to surgery and anaesthesia, a serving of the formulation of the present invention is ingested by the patient. Ideally, the formulation is ingested at least 90 minutes before surgery and anaesthesia. Preferably the ingestion is about 120 minutes before surgery and anaesthesia. This time will be sufficient for the formulation to reduce the side effects of fasting such as hunger and thirst and avoid the irritability prior to surgery and anaesthesia that results from other side effects such as headaches and light headedness.

A serving of the formulation may include at least 25 grams of maltodextrin. Preferably, a serving may include maltodextrin in the range of 25 to 75 grams; more preferably a serving may include any of 25, 30, 35, 40, 45, 50, 55, 60, 65, 70 or 75 grams of maltodextrin. More preferably, a serving may include 50 grams of maltodextrin. One or two servings of the formula may be consumed depending on the patient's level of hunger and/or thirst. However, 25 to 75 g of maltodextrin should be consumed; preferably 50 grams may be served prior to surgery and anaesthesia. This amount of maltodextrin may be beneficial for reducing insulin resistance post-operatively.

The exclusion of fructose from the preoperative carbohydrate drink is to minimise postoperative insulin resistance. Fructose may contribute to insulin resistance so that avoiding fructose may make the formulation of the present invention more effective at preventing insulin resistance than other surgery specific preoperative carbohydrate drinks.

Preferably a first ingestion of the formulation is about at least 4 hours before surgery and a second ingestion of the formulation is about at least 90 minutes prior to surgery. More preferably the formulation is ingested at a time prior to administration of anaesthesia so that the stomach is close to empty when anaesthesia commences.

Preferably patients who are awaiting surgery or surgical review may be offered approximately 200 mls of the formulation every hour. This may be sufficient to alleviate feelings of hunger and thirst whilst optimally preparing the patient for surgery and anaesthesia.

In another embodiment the method includes instructions for preparing for surgery. The instructions may be provided on a fasting card to the patient during fasting. The fasting card may provide dosing and schedule times for ingestion of the formulation prior to surgery. This will alleviate confusion as to the types of fluids that can be ingested and the timing for the ingestion that will best prepare the patient for pre- or post-surgery. If the patient is better prepared pre- or post-surgery it is more likely that the patient will perform better pre- or post-surgery and recovery times will be shortened.

In another aspect there is provided a fasting card when used by a patient during fasting or prior to surgery said fasting card providing dosing and schedule times for ingestion of a formulation comprising maltodextrin and which is devoid of simple sugars.

In one embodiment the fasting card refers to a formulation according to the present invention.

The present invention will now be more fully described by reference to the following non-limiting Examples.

### Examples

### Example 1: Fasting Formulation

A formulation comprising maltodextrin for use as a pre-operative formulation was prepared so that 50 g maltodextrin was administered in one serving to a patient prior to surgery. The following formulation was prepared:

| | | |
|---|---|---|
| Maltodextrin (14%) | 14 | g |
| Water | Approx. 100 | mls |
| Other ingredients including acid (330), | 20 | mg |
| acidity regulators* - Sodium citrate and | | |
| potassium citrate (331, 332), natural | | |
| flavours (mango, coconut), natural colours | | |
| (beta-carotene, anthocyanins) | | |

The formula has the following characteristics:

| | Osmolarity | | Sodium | | |
|---|---|---|---|---|---|
| | (mOsm) per | Energy (kj) | (mg) per | Complex | Simple |
| pH | kg | per 100 mls | 100 mls | carbohydrate | sugars |
| 4.1 | 174 mOsm/kg | 250 | 20 | 100% | 0% |

### Example 2: Reduction of Gastrointestinal Discomfort

Patients were tested for their gastrointestinal response to various pre-operative drinks compared to the formulation of the present invention.

A formulation was prepared in accordance with Example 1 (Dex™) and compared against other available pre-operative formulations Preop™ and ClearFast™.

Preop™ is a 0.5 kcal/ml, clear, non-carbonated, lemon flavoured, iso-osmolar carbohydrate drink. It contains water, maltodextrin, fructose, tri potassium citrate, tri sodium citrate, acidity regulator (citric acid), flavour (lemon), sweetener (acesulfame K), sweetener (sodium saccharine).

ClearFast™ is a clear, carbohydrate rich drink, made for patients fasting before surgery or procedures. It contains filtered water, maltodextrin, crystalline fructose, L-Citrulline, Natural Flavours, Sodium citrate, Citric acid, Malic acid, Stevia Rebaudiana, Monopotassium Phosphate, Zinc Sulphate, Vitamin A Palmitate, Sodium Selenite complex carbohydrates, electrolytes, Vitamin A, Selenium, Zinc, and L-citrulline.

50 patients fasted for 6 hours and then consumed 600 mls of Dex™ over a 2 minute period. 2 patients complained of stomach cramps.

50 patients fasted for 6 hours and then consumed 600 mls of a competitor drink (Preop™ (25 patients), ClearFast™ (25 patients)) that contained 6 g Fructose. The drink was consumed over 2 mins.

In the Preop™ group 4 patients complained of GI upset, and in the ClearFast™ group 5 patients complained of GI upset.

In total 9 patients complained of GI upset when drinking a known pro-operative drink containing fructose compared to Dex™.

### Example 3: Use of Formulation Dex™ Prior to Surgery

The pre-operative formulation is ingested prior to surgery. However, a preferred regime of fasting is adhered to ensuring that the body is ready for surgery.

(a) For Morning Surgery

Patients are requested to stop eating at midnight and before retiring for the evening before surgery to induce fasting. Once awake, preferably in the morning they are encouraged to consume one serving of the formulation prepared in accordance with Example 1.

The patient is then encouraged to consume a second serving of the formulation from Example 1 at approximately 90 mins and up to 2 hrs before admission time.

Alternatively, both servings may be consumed at this time.

The patient is also encouraged to consume the formulation on an as needs basis prior to surgery.

(b) For Afternoon Surgery

Patients are requested to stop eating at about 7.00 am to induce fasting prior to the afternoon surgery. At approximately 11.00 am they are encouraged to consume one serving of the formulation prepared in accordance with Example 1.

The patient is then encouraged to consume a second serving of the formulation from Example 1 at approximately 90 mins and up to 2 hrs before admission time. Alternatively, both servings may be consumed at this time.

Best results are obtained when at least two servings of the the formulation are consumed before surgery.

The patient is also encouraged to consume the formulation on an as needs basis prior to surgery.

For pediatric patients, the same regime may be applied except that the first serving is replaced with 10 ml/kg (up to a maximum of 200 mls) and the second serving is replaced with 10 ml/kg (up to a maximum of 200 mls).

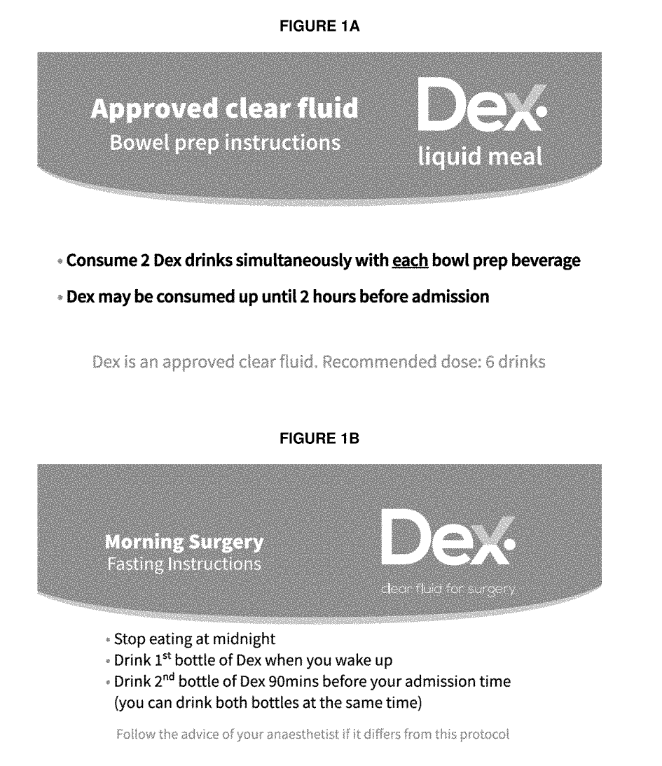

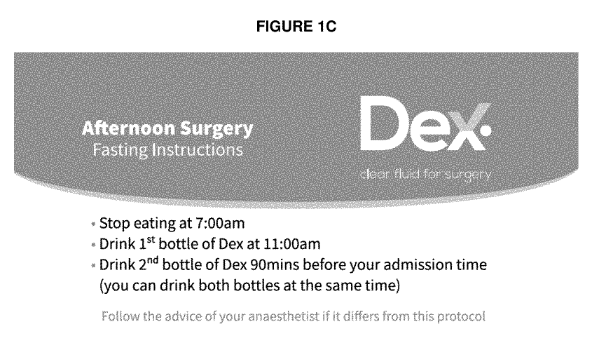

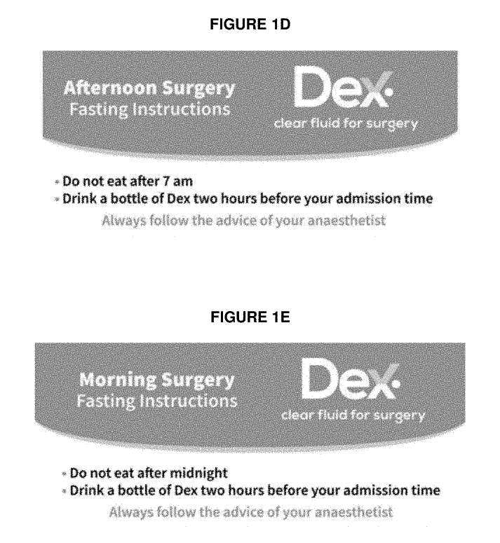

Simple instructions for the use of the formulation may be provided on Fasting Cards that are easily provided to the patients with simple instructions for the use of the formulation prior to surgery (see FIG. 1). The Fasting Cards when used along with the formulation alleviates confusion for the types of fluids that a patient can ingest prior to surgery and also provides an indication of the schedule for ingestion to best prepare the patient pre- and post-surgery.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

1. A formulation when used to reduce side effects of fasting comprising maltodextrin and is devoid of simple sugars.

2. A formulation according to claim 1, wherein the side effect is gastrointestinal (GI) discomfort.

3. A formulation according to claim 2, wherein the GI discomfort arises during or after fasting.

4. A formulation according to claim 1 when used during or after fasting.

5. A formulation according to claim 1 wherein the formulation is devoid of fructose.

6. A formulation according to claim 1 wherein the formulation is a beverage.

7. A formulation according to claim 1 when used as a preoperative formulation.

8. A formulation according to claim 1 when used prior to administration of anaesthesia.

9. A formulation according to claim 1 when used to satisfy potential hunger and/or thirst.

10. A formulation according to claim 1 having an osmolality in the range of 130 mOsm/kg to 180 mOsm/kg, optionally at least 149 or 174 mOsm/kg.

11. A formulation according to claim 1 wherein the pH is at least 4.0, optionally 4.2.

12. Use of maltodextrin in the absence of simple sugars in the preparation of a formulation which is devoid of simple sugars to reduce side effects of fasting.

13. A use according to claim 12, wherein the side effect is GI discomfort.

14. A use according to claim 12 wherein the GI discomfort arises during or after fasting.

15. A use according to claim 12 to reduce side effects during or after fasting.

16. A use according to claim 12 wherein the formulation is devoid of fructose.

17. A use according to claim 12 wherein the formulation is a beverage.

18. A use according to claim 12 wherein the fasting is prior to surgery.

19. A use according to claim 12 wherein the fasting is prior to administration of anaesthesia.

20. A use according to claim 12 to further satisfy potential hunger and/or thirst during the fasting.

21. A use according to claim 12 wherein the formulation has an osmolality in the range 130 mOsm/kg to 180 mOsm/kg, optionally of at least 149 or 174 mOsm/kg.

22. A use according to claim 12 wherein the formulation has a pH of at least 4.0, optionally 4.2.

23. A method for preparing a patient for surgery, said method comprising the patient:
ceasing eating for a time prior to surgery to induce fasting in the patient; and
ingesting a formulation comprising maltodextrin and which is devoid of simple sugars during the fasting at about at least 90 minutes prior to surgery.

24. A method for preparing a patient for surgery, said method comprising the patient:
ceasing eating for a time prior to surgery to induce fasting in the patient; and
ingesting a formulation according to claim 1 during the fasting at about at least 90 minutes prior to surgery.

25. A method according to claim 23 wherein the time prior to surgery is at least 12 hours.

26. A method according to claim 23 wherein a first ingestion of the formulation is about at least 4 hours before surgery and a second ingestion of the formulation is about at least 90 minutes prior to surgery.

27. A method according to claim 23 further ingesting the formulation to satisfy hunger and/or thirst during the fasting.

28. A method according to claim 23 wherein the formulation comprising maltodextrin is devoid of fructose.

29. A method according to claim 23 wherein the fasting is prior to administration of anaesthesia.

30. A method according to claim 29 wherein the formulation is ingested at a time prior to administration of anaesthesia so that the stomach is close to empty when anaesthesia commences.

31. A method according to claim 23 wherein instructions for preparing for surgery are provided on a fasting card to the patient during fasting said fasting card providing dosing and schedule times for ingestion of the formulation prior to surgery.

32. A fasting card when used by a patient during fasting or prior to surgery said fasting card providing dosing and schedule times for ingestion of a formulation comprising maltodextrin and which is devoid of simple sugars.

33. A fasting card when used by a patient during fasting or prior to surgery said fasting card providing dosing and schedule times for ingestion of a formulation according to claim 1.
